(19) United States
(12) Patent Application Publication
(10) Pub. No.: US 2005/0176080 A1
(43) Pub. Date: Aug. 11, 2005

(54) Hapten, immunogens and derivatives of ascomycin useful for preparation of antibodies and immunoassays

(76) Inventors: Vani Bodepudi, San Ramon, CA (US); Lisa Charter, Fremont, CA (US); Rueyming Loor, Danville, CA (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/049,547

(22) Filed: Feb. 2, 2005

Related U.S. Application Data
(60) Provisional application No. 60/543,380, filed on Feb. 10, 2004.

(51) Int. Cl. G01N 33/53; G01N 33/537; G01N 33/543; C07K 16/18; C07K 14/47

(52) U.S. Cl. 435/7.92; 530/387.1; 530/409

(57) ABSTRACT

The invention teaches derivatives of ascomycin and methods of preparing immunogens and other conjugates useful in immunoassays for quantitatively measuring concentrations of tacrolimus in patient specimens. Antibodies produced from the disclosed immunogens capable of binding to tacrolimus with cross-reactivity of no more than 5% with each of 15-O-demethyl tacrolimus, 31-O-demethyl tacrolimus, and 13,31-O-didemethyl tacrolimus, less than 40% with 13-O-demethyl tacrolimus, and less than 1% with cyclosporin, rapamycin, mycophenolic acid, prednisone, hydrocortisol, and prednisolone are described. Further, immunoassays for measuring the concentration of tacrolimus using such antibodies are taught.

### CROSS REFERENCE TO RELATED APPLICATION

The present application is based on and claims priority from U.S. Provisional Patent Application Ser. No. 60/543,380 filed on Feb. 10, 2004.

### FIELD OF THE INVENTION

The invention relates to prepared derivatives of ascomycin, including immunogens used to stimulate production of antibodies capable of specifically binding tacrolimus and other conjugates, useful in immunoassays for quantitatively measuring concentrations of tacrolimus in patient specimens.

### BACKGROUND OF THE INVENTION

Tacrolimus, a macrolide antibiotic described in U.S. Pat. No. 4,894,366 to Okuhara et al., incorporated herein, is a metabolic product of the fungus Streptomyces tsukubaensis with potent immunosuppressive function. Also referred to as FR-900506 and FK506, tacrolimus is prescribed for patients following organ transplantation, particularly kidney or liver transplantation. At the intracellular level, tacrolimus binds a family of proteins, in particular, FKBP12. A pentameric complex comprising tacrolimus, FKBP12, calcium, calcineurins A and B, and calmodulin inhibits phosphatase activity of calcineurin, a component required for activation and transport of transcriptional factors into the cell nucleus of T lymphocytes (T cells). Consequently, gene expression in T cells is impaired, especially for cytokines such as interleukin-2, resulting in an immunosuppressive effect. However, the therapeutic value of the immunosuppressive effect of tacrolimus can be lost due to the toxicity of tacrolimus beyond therapeutic concentrations. Thus, measurement of tacrolimus concentrations in whole blood offers a useful approach to optimize immunosuppressive effect and minimize adverse side effects.

Ascomycin, described in U.S. Pat. No. 3,244,592 to Arai, incorporated herein, is a metabolic product of Streptomyces hygroscopicus var. ascomyceticus. Ascomycin, also referred to as FR-900520 and FK520, is substantially identical in chemical structure to tacrolimus, but ascomycin has an ethyl moiety instead of an allyl moiety at position 21. FIG. 1 depicts the structure of tacrolimus and ascomycin. The chemical distinction is reflected by differences in the physical properties between tacrolimus and ascomycin. For example, the melting point of tacrolimus as 127-129° C., while ascomycin's melting point is 159-161° C. Moreover, differences in solubility characteristics and pharmacologic efficacy of tacrolimus and ascomycin have led to differences in commercial pharmaceutical products and treatment protocols such that the drugs are not necessarily interchangeable in terms of medical applications. The FDA approved intravenous and oral uses of tacrolimus, but not ascomycin, as immunosuppressive treatments for the prevention of organ rejection after allogenic liver or kidney transplantation.

Immunoassays are becoming increasingly popular as methods for monitoring levels of therapeutic drugs in fluid specimens. A particular challenge in the development of immunoassays is the production of an antibody to the target drug since many therapeutic drugs are not antigenic. Lack of antigenic properties is especially problematic when the target drug is itself an immunosuppressant. Generally, the drug must be modified to make an antigenic derivative, yet the antibody produced to the antigenic derivative must be able to recognize the drug as it is contained in the fluid specimen to be tested with an appropriately useful degree of sensitivity.

Sensitivity is not the only concern. Often metabolites, with little or no therapeutic value, of the drug to be monitored exist at varying levels, creating specificity issues. Preferably, for use in quantitative assays, an antibody to a particular therapeutic drug has minimal, if any, cross-reactivity with metabolites of the drug. In the case of tacrolimus, metabolites include 13-O-demethyl tacrolimus, 15-O-demethyl tacrolimus, 31-O-demethyl tacrolimus, 13,31-O-didemethyl tacrolimus, 15,31-O-didemethyl tacrolimus, and 12-hydroxy tacrolimus. Further, an antibody useful in measuring a particular therapeutic drug has minimal, if any, cross-reactivity with commonly co-administered drugs.

Previous reports describe antibodies developed to bind tacrolimus (FR-900506) and immunoassays for quantitative detection of tacrolimus. U.S. Pat. No. 5,532,137 to Niwa et al., incorporated herein in entirety, teaches modification of the hydroxyl group on the cyclohexyl moiety of tacrolimus for the formation of an antigenic derivative. In particular, tacrolimus is converted to a half ester of a dicarboxylic acid (hemisuccinate). It is then reacted with N-hydroxysuccinimide in the presence of a condensing agent (dicyclohexylcarbodiimide) to form an active ester and, finally, coupled to an antigenic protein such as bovine serum albumin. Cross-reactivities of antibodies developed using the immunogen taught by Niwa et al. with either tacrolimus metabolites or with commonly co-administered immunosuppressants are not disclosed.

U.S. Pat. No. 6,635,745 to Sedrani et al. teaches the use of immunogenic conjugates of the drug rapamycin (also referred to as “sirolimus”) to produce antibodies that bind rapamycin and have greater than 50% cross-reactivity with FK-506 (tacrolimus).

International application WO 01/09190 (Kasper et al.), incorporated herein in entirety, teaches an antigenic derivative formed by modifying tacrolimus at position 22 with a carboxymethyl oxime followed by coupling to an antigenic protein such as keyhole limpet hemocyanin. A preferred monoclonal antibody reported by Kasper et al., designated 1H6, shows cross-reactivity greater than or equal to 5% with 13-O-demethyl tacrolimus, 15-O-demethyl tacrolimus, and 31-O-demethyl tacrolimus; in particular, cross-reactivity of monoclonal antibody 1H6 with 13-O-demethyl tacrolimus is reported to be 78%. Cross-reactivity of monoclonal antibody 1H6 with commonly co-administered immunosuppressants is not disclosed.

Applicants realized that use of an antigenic derivative of ascomycin to stimulate antibody production might yield an antibody with suitable sensitivity to tacrolimus, while minimizing cross-reactivity to tacrolimus metabolites and commonly co-administered immunosuppressants and, therefore, be useful in developing immunoassays to monitor tacrolimus concentrations in patient specimens.

### SUMMARY OF THE INVENTION

In one aspect, the present invention comprises derivatives of ascomycin prepared by reacting the keto group at position 22 with carboxymethoxylamine to form an oxime. In another aspect of the present invention, the oxime is further modified by conjugation to an antigenic molecule, such as a protein or poly(amino acid), to form an immunogen for the production of antibodies with sensitivity and specificity for tacrolimus. In another aspect, the present invention comprises production of antibodies useful for measuring concentrations of tacrolimus in patient specimens wherein the antibodies are produced by using an antigenic derivative of ascomycin as an immunogen. In yet another aspect, the present invention teaches conjugates formed by coupling a carboxymethyl oxime derivative of ascomycin with a labeling molecule, such as an enzyme, enzyme fragment, enzyme donor, biotin, fluorescein, and the like. Also, the present invention encompasses a quantitative immunoassay for tacrolimus that utilizes an antibody reagent developed by using an antigenic derivative of ascomycin as an immunogen.

### BRIEF DESCRIPTION OF FIGURES

FIG. 1 shows the chemical structure of ascomycin and tacrolimus.

FIG. 2 shows the chemical structure of an exemplary derivative of ascomycin as taught in Example 1.

FIG. 3 represents the chemical structure of an exemplary immunogenic derivative of ascomycin as described in Example 2.

FIG. 4 shows the chemical structure of an exemplary adduct of ascomycin as described in Example 3.

FIG. 5 represents the chemical structure of an exemplary labeled conjugate as described in Example 4.

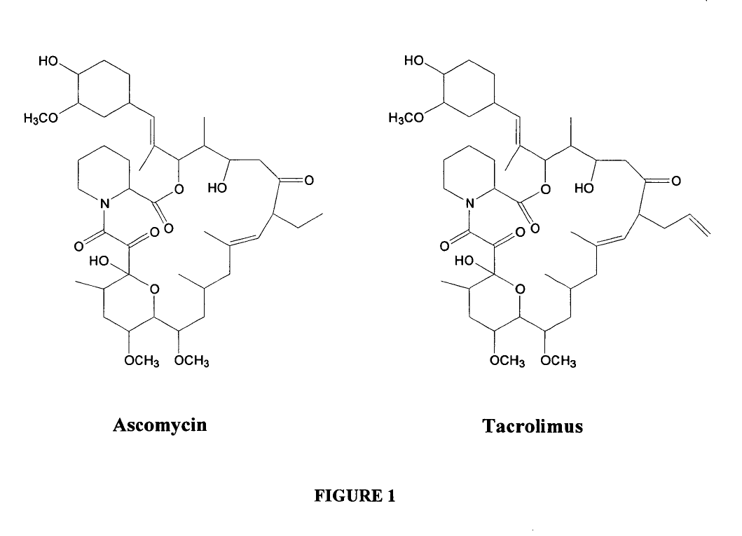

### DETAILED DESCRIPTION OF THE INVENTION

The present invention, in all of its interrelated embodiments, involves preparation of a carboxymethyl oxime derivative of ascomycin which can then be used to form immunogens by coupling the derivative to immunogenic poly(amino acids) or other antigenic carriers. The immunogens are utilized to produce antibodies with sufficient sensitivity and specificity for tacrolimus for use as reagents in quantitative immunoassays for tacrolimus. Alternatively, carboxymethyl oxime derivatives of ascomycin are used to form enzyme donor or other labeled conjugates useful as detection agents in immunoassays for tacrolimus.

In one embodiment of the present invention, ascomycin is converted to a carboxymethyl oxime derivative at position 22 by reacting the keto group with carboxymethoxylamine, as described in Example 1, and then further modified by conjugation to an immunogenic molecule including, but not limited to, keyhole limpet hemocyanin (KLH), bovine serum albumin (BSA), and ovalbumin, as described in Example 2.

In another aspect of the invention, the resulting immunogenic conjugate of ascomycin is useful for developing antibodies capable of specifically binding to tacrolimus. The term “antibody” as used herein refers to polyclonal and monoclonal antibodies and related antigen recognition units, including fragments and derivatives of immunoglobulin molecules. One method of producing antibodies is to administer the immunogen, generally combined with an adjuvant such as Freund's, in a series of injections to a host animal for the purpose of inducing an immunologic response. Such methods are well known to those skilled in the art. Methods for producing monoclonal antibodies were first described by Kohler and Milstein (Nature, Vol 256, pp 495-497, 1975; incorporated herein in its entirety) and have been modified several times since the appearance of that publication. For hybridoma technology, the reader is directed generally to U.S. Pat. Nos. 4,491,632, 4,472,500, and 4,444,887, and Methods in Enzymology, 73B:3 (1981); each is incorporated herein in entirety. Since the particular method is not critical, any proven method can be used to produce an antibody using the immunogen described herein.

Antibodies obtained using any of the aforementioned techniques are screened or purified not only for their ability to bind specifically with tacrolimus, but also for low cross-reactivity with potential interfering substances. “Cross reactivity” is determined in a quantitative immunoassay by establishing a standard curve using known dilutions of tacrolimus or ascomycin. The standard curve is then used to calculate the apparent concentration of the interfering substance present in various known amounts in samples assayed under substantially similar conditions. The percent (%) cross-reactivity is the measured concentration divided by the tested concentration multiplied by 100.

Antibodies are generally screened for cross-reactivity with other pharmaceutical compounds that subjects may be taking collaterally and/or with molecules that have structural resemblance to tacrolimus, such as metabolites. Antibodies useful in quantitative immunoassays preferably have levels of cross-reactivity of no more than 5%. With regards to an assay for tacrolimus, other immunosuppressants, such as cyclosporine, sirolimus, mycophenolic acid, prednisone, hydrocortisol, and prednisolone, are of particular concern. Also, metabolites of tacrolimus, including 13-O-demethyl tacrolimus, 15-O-demethyl tacrolimus, 31-O-demethyl tacrolimus, and 13,31-O-didemethyl tacrolimus present potential cross-reactivity concerns.

In another embodiment of the invention, the carboxymethyl oxime of ascomycin described herein is used to form conjugates with other molecules or solid matrices that are useful as labeling compounds in immunoassays. Such conjugates include, but are not limited to, enzymes, enzyme donors, enzymes fragments, biotin, fluorescent molecules, radioisotopes, metal sols, latex particles, membranes, polymer surfaces, and the like. Use of a linker between the ascomycin derivative and the labeling molecule or particles is generally preferred. Linkers can be of different lengths and different structures, as is known in the art. The reader is referred generally to Hermanson, G. T., “Bioconjugate Techniques”, Academic Press: New York, 1996; and “Chemistry of Protein Conjugation and Cross-linking” by S. S. Wong, CRC Press, 1993, incorporated herein. Methods for preparing a maleimidoethylamine adduct and conjugating it to an enzyme donor fragment are described in Examples 3 and 4.

The present invention also anticipates use of antibodies prepared from ascomycin-containing immunogens, as well as other conjugates prepared from ascomycin, as reagents for performing immunoassays for the quantitative detection of tacrolimus. In testing for drugs, immunoassays, particularly competitive binding immunoassays, have enjoyed increasing popularity. In competitive binding immunoassays, an analyte, for example a target drug, in a specimen competes with a labeled reagent, or analyte analog, or a detectable tracer, for limited number of receptor binding sites on antibodies capable of specifically binding the analyte and/or analyte analog. Enzymes, including peroxidase, phosphatase, and β-galactosidase, and fluorescent molecules or particles are commonly used as labeling substances, and radioisotopes remain in use as labels. The amount of target analyte in a specimen determines how much labeled analyte or analyte analog becomes bound to the antibodies specific for the target analyte.

A preferred form of immunoassay is cloned enzyme donor immunoassay or CEDIA® (trademark of Roche Diagnostics), based upon the reassociation of enzymatically inactive polypeptide fragments of β-galactosidase. CEDIA® has proven to be a highly accurate method for quantitative measurements of therapeutic drugs and drugs of abuse. CEDIA is the subject of several patents including U.S. Pat. No. 4,708,929 which claims competitive homogeneous assay methods, U.S. Pat. No. 5,120,653 claiming a recombinant DNA sequence for coding the enzyme donor fragment and a host for such a vector, U.S. Pat. No. 5,604,091 which claims amino acid sequences of the enzyme donor fragment, and U.S. Pat. No. 5,643,734 which teaches and claims kits for CEDIA assays. All of the above patents are incorporated in entirety herein. In particular, β-galactosidase enzyme donor polypeptide fragment combines with a β-galactosidase enzyme acceptor fragment to form active β-galactosidase enzyme. The active enzyme complex is capable of transforming a substrate into a product that is differentially detectable. Usually, the product is a different color from the substrate and is quantified using spectrophotometric methods. Conjugating a hapten or other small analyte or analyte analog to the enzyme donor fragment at certain sites does not affect the ability to form active enzyme by the complementation reaction and does not affect the rate of enzymatic activity when in the presence of a substrate for β-galactosidase. However, when the enzyme donor-hapten conjugate is bound by the anti-analyte antibody, for example, when little or no analyte is present in a specimen being tested, the complementation reaction is inhibited, reducing the amount of active enzyme present in the reaction mixture. Hence, the enzyme-catalyzed reaction rate is decreased under such conditions. In contrast, when the specimen tested contains significant concentrations of a target analyte, it competes with the enzyme donor-hapten for binding sites on the anti-analyte antibody, thereby increasing the amount of active enzyme formed by complementation reaction. Therefore, the enzyme-catalyzed reaction rate is directly proportional to the concentration of target analyte present in the specimen tested.

A preferred β-galactosidase enzyme donor is ED28, a polypeptide containing residues 6-45 of β-galactosidase, with cysteines at postions 1 and 46 (relative to the numbering of the original β-galactosidase fragment). Typical linker groups used are maleimide adducts as described in Examples 3 and 4.

Preferred substrates for use in immunoassays utilizing β-galactosidase include those described in U.S. Pat. Nos. 5,032,503; 5,254,677; 5,444,161; and 5,514,560, incorporated herein. Chlorophenol-red-β-D-galactopyranoside is an exemplary substrate.

### EXAMPLE 1

### Preparation of Ascomycin-CMO Hapten

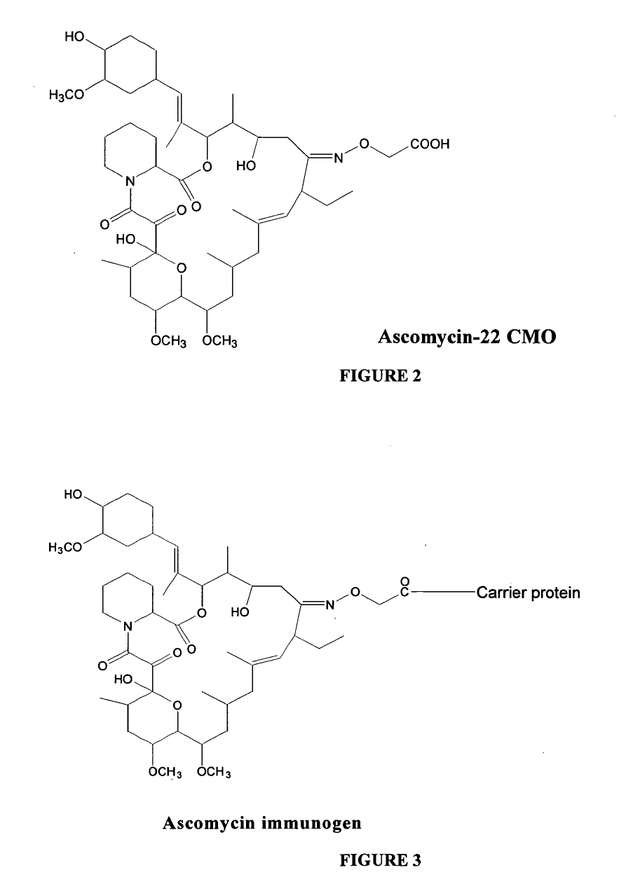

To a stirred solution of ascomycin (30 mg, 0.038 mmol) in 3 ml of methanol were added sodium acetate (8 mg, 0.095 mmol) and carboxymethoxylamine hemihydrochloride (16.5 mg, 0.076 mmol). The resulting reaction mixture was stirred at room temperature for 24 hrs and monitored by thin layer chromatography (silica gel; methanol:chloroform, 5:95) to determine completion of the reaction. Methanol was removed using a roto-evaporator and diluted with 20 ml of water. The pH of the reaction mixture was adjusted to 5.0 with 1 N HCl and extracted with dichloromethane, 3×25 ml. The dichloromethane layer was washed with water, brine and dried over anhydrous magnesium sulfate. The solvent was removed under reduced pressure to yield 29 mg of ascomycin-carboxymethyl oxime (ascomycin-CMO) as a white crystalline solid. The structure of ascomycin-CMO prepared according to the exemplary method is shown in FIG. 2.

### EXAMPLE 2

### Preparation of Immunogen from Ascomycin-CMO Hapten

To a stirred solution of ascomycin-CMO (4.3 mg, 0.00497 mmol) in 0.5 ml of dimethyl formamide (DMF) was added 1-ethyl-3-(3-dimethylaminopropyl)carbodiimide hydrochloride (EDAC) (3 mg, 0.0156 mmol) and N-hydroxy-5-norbornene-2,3-dicarboximide (NHDC) (2.5 mg, 0.0137 mmol). The reaction mixture was stirred at room temperature for 4 hours and monitored by thin layer chromatography (silica gel; methanol:chloroform, 5:95). The resulting ascomycin-CMO-NHDC ester was reacted with a substance having antigenic carrier protein properties, such as bovine serum albumin (BSA), keyhole limpet hemocyanin (KLH), ocular lens proteins, egg ovalbumin, lipoproteins, and the like, or any protein fragment thereof. FIG. 3 shows the structure of an immunogen according to an embodiment of the present invention.

As a further example, to a solution of KLH (20 mg) in 0.1 M PBS, pH 7.0 (2.4 ml) and DMF (0.1 ml) was added 0.5 ml of the above-described ascomycin-CMO-NHDC ester solution. The reaction mixture was stirred at room temperature for 15 hrs and was purified by dialyzing against PBS-20% DMF, PBS two times using 2 L for each dialysis, for a minimum of 7 hours to give 20 mg of ascomycin-CMO-KLH immunogen.

### EXAMPLE 3

### Preparation of Ascomycin-CMO-MEA Adduct

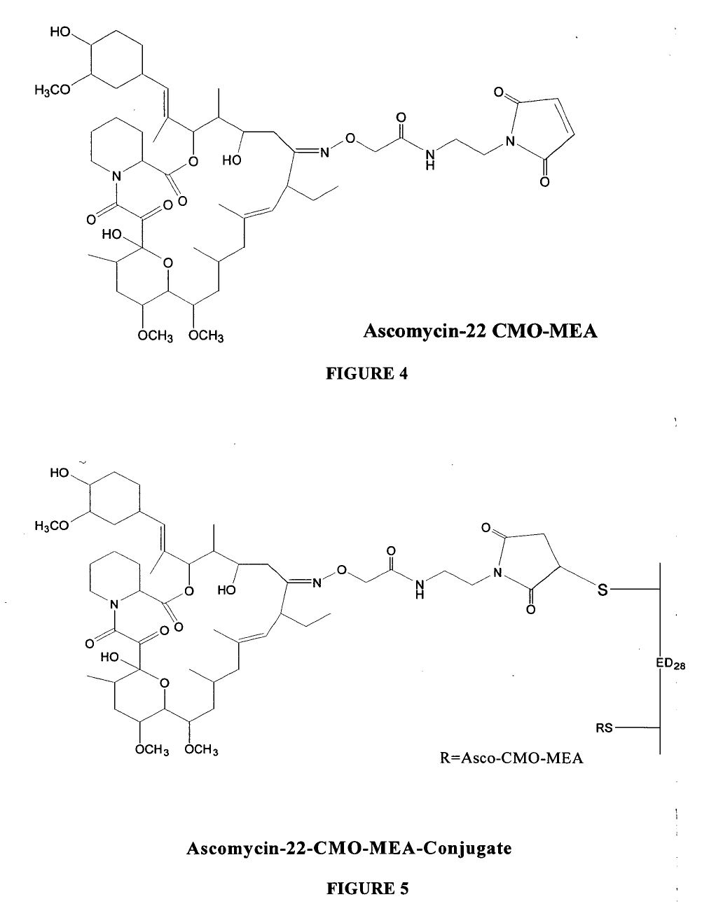

To a stirred solution of ascomycin-CMO (10 mg, 0.01157 mmol) in DMF (1 ml) were added EDAC (6.628 mg, 0.0347 mmol) and NHDC (6.32 mg, 0.0347 mmol). The reaction mixture was stirred at room temperature for 3 hours and monitored by thin layer chromatography (silica gel; methanol:chloroform, 5:95). Maleimidoethylamine (MEA) hydrochloride (6.13 mg, 0.0347 mmol) was added to the reaction mixture and the reaction mixture pH was adjusted to 8.0 with triethyl amine. The solution was stirred at room temperature for 30 minutes. The reaction mixture was purified by HPLC to give 4.5 mg of ascomycin-CMO-MEA as a white solid. The structure of an ascomycin-CMO-MEA adduct prepared according to the exemplary method is shown in FIG. 4.

### EXAMPLE 4

### Preparation of Ascomycin-CMO-MEA-Enzyme Donor Conjugate

To a solution of 1 mg of enzyme donor (ED28) enzyme in 0.76 ml of PBS buffer was added a solution of 0.6 mg of ascomycin-CMO-MEA in 0.23 ml of acetonitrile. The solution was mixed on a vortex mixer for 5 seconds and kept at 4° C. for 45 minutes. Ascomycin-CMO-MEA-ED28 conjugate was purified by HPLC and quantified using standard spectrophotometric methods known in the art. FIG. 5 shows the structure of an enzyme donor conjugate prepared according to the exemplary method described.

### EXAMPLE 5

### Preparation of Other Ascomycin-CMO-MEA Conjugates

Ascomycin-CMO-MEA adduct as prepared above can be used, alternatively, to form conjugates with other molecules including, but not limited to, biotin, horseradish peroxidase or other enzymes, fluorescent labels, chemiluminescent labels, bioluminescent labels, radioactive labels, and dyes, or linked to solid matrices such as metal sols, latex particles, fluorescent particles, membranes, polymer surfaces and the like. Such conjugates are formed by any number of routine procedures well known to those skilled in the art. It is possible to make conjugates from ascomycin that are useful for a variety of immunoassays, including FPIA, ELISA, lateral flow immunoassays and immunoturbidimetric assays.

### EXAMPLE 6

### Preparation of Antibodies Using Ascomycin-CMO-Carrier Immunogen

The immunogen of Example 2 was administered to mice in a series of injections as is routine in the art. Spleens of immunized mice were fused with an immortal non-producing myeloma to develop hybridoma cell lines capable of shedding antibodies into cell culture supernatant. A primary screening of the supernatants was performed to evaluate the ability of the antibodies to bind to enzyme-donor conjugate, as prepared above, and inhibit enzyme complementation with the enzyme-acceptor fragment. The number of inhibition-positive clones were narrowed by performing further screening assays to determine whether free tacrolimus modulates or competes with the enzyme-donor conjugate for the antibody using standard CEDIA protocols as cited above and incorporated herein. For the purposes of the present invention, one hybridoma cell line, designated 28E2, was selected for production of a monoclonal antibody capable of specifically binding tacrolimus without significant cross-reactivity to other immunosuppressants or to tacrolimus metabolites, except 13-O-demethyl tacrolimus.

Alternatively, supernatants may be tested for the production of antibodies by any number of methods well known to those skilled in the art, including, but not limited to ELISA or other standard immunoassays. Clones showing desired sensitivity and specificity are selected for further propagation. A supply of monoclonal antibody from the selected clone is then purified from a volume of culture supernatant or from ascites fluid of suitably prepared host animals injected with cells from the clone. Supernatants and ascites may be used unprocessed or, preferably, purified by biochemical means such as ammonium sulfate precipitation, gel filtration chromatography, or ion exchange chromatography, etc. The monoclonal antibodies produced are suitable for a variety of immunoassay formats.

### EXAMPLE 7

### CEDIA Tacrolimus Assays, Reagents and Kits

The CEDIA assay uses a two-point linear calibration from 0 to 30 nanograms of drugs per milliliter of whole blood and is applicable for use with automated clinical chemistry analyzers, such as the Hitachi 911 or 917 or comparable analyzers. Monoclonal antibody produced by hybridoma line 28E2 was selected for use in the assay. The assay is based upon the competition of tacrolimus in the biological sample with a conjugate comprising the inactive enzyme-donor (ED) fragment of β-galactosidase coupled to tacrolimus or ascomycin or an analog thereof, for binding to the antibody reagent. If tacrolimus is present in the sample, it binds to the antibody, leaving the ED portion of the competing conjugate free to restore enzyme activity upon complementation reaction with enzyme acceptor (EA) fragments in the assay reaction mixture. The active enzyme is then capable of producing a quantifiable reaction product when exposed to appropriate substrate. If drug is not present in the sample, the antibody binds to the ED-drug conjugate inhibiting association of the ED fragments with the EA fragments, thus inhibiting restoration of enzyme activity. The amount of reaction product and resultant absorbance change are proportional to the amount of drug in the sample.

Kits for performing CEDIA assays have been generally described in other patents cited herein. As an example, CEDIA kits for measuring tacrolimus concentrations in fluid specimens, particularly whole blood, contain β-galactosidase enzyme acceptor (EA) reagent comprising EA lyophilized in a buffered salt solution, preferably at a concentration of about 0.118 grams of EA per liter of buffered salt solution prior to lyophilization. A preservative such as sodium azide is beneficial to increase the shelf life. Also included is an EA reconstitution buffer that includes antibodies to tacrolimus, for example the monoclonal antibody produced by hybridoma line 28E2 at approximately 0.7 micrograms per milliliter. Preferred buffers include PIPES, MOPS, HEPES, TES or Tris.

The enzyme donor (ED) fragment conjugated to ascomycin is supplied in the kit as a separate reagent lyophilized along with the substrate. Chlorophenol-red-β-D-galactopyranoside at a concentration of about 10 nM (about 119 nanogram per ml) is a preferred substrate. Also, stabilizers, such as bovine serum albumin fragments, and preservatives, such as sodium azide, are beneficial in extending the shelf life. The ED reagent is reconstituted with ED reconstitution buffer comprising a buffer such as MES, plus a non-ionic detergent (Tween 20, NP-40, etc.), and preservative. Additional components of the kit include instructions for performing the assay. Optionally, the kit may include calibrators, for example, at least one with no ascomycin or tacrolimus (0 ng/ml ascomycin or tacrolimus) and one in a higher concentration range (>30 ng/ml ascomycin or tacrolimus) and tacrolimus and/or ascomycin controls comprising known concentrations of the drugs. Calibrators and/or controls are included in kits or provided as separate components.

### EXAMPLE 8

### Intra- and Inter-assay Precision (Reproducibility) of CEDIA Assay

Intra- and inter-assay precision studies were conducted using whole blood samples from patients treated with tacrolimus and from spiked samples as measured in the CEDIA Assay described in Example 7. Assay precision (intra- and inter-assay reproducibility) was determined using 21 individual runs over a period of 11 days as described in the NCCLS replication experiment, EP5-A. Results of intra-assay and total assay precision studies are shown in Table 1.

TABLE 1
Intra- and Total Assay Precision

| | | | S.D. | CV % | S.D. | CV % |
|---|---|---|---|---|---|---|
| Sample | n | mean | (within) | (within) | (total) | (total) |
| Patient 1 | 126 | 6.30 | 0.43 | 6.80 | 0.49 | 7.80 |
| Patient 2 | 126 | 9.23 | 0.43 | 4.68 | 0.56 | 6.06 |
| Patient 3 | 126 | 15.18 | 0.47 | 3.12 | 0.65 | 4.26 |
| Spiked 1 | 126 | 5.33 | 0.32 | 5.97 | 0.48 | 8.90 |
| Spiked 2 | 126 | 10.54 | 0.35 | 3.28 | 0.49 | 4.69 |
| Spiked 3 | 126 | 21.01 | 0.43 | 2.04 | 0.72 | 3.40 |

### EXAMPLE 9

### Recovery in CEDIA Tacrolimus Assay

Recovery was tested in two different experiments. First, linearity of the assay was tested using a tacrolimus-free whole blood sample spiked with tacrolimus at a concentration of 28.9 ng/ml. The spiked specimen was then used straight (undiluted) or was diluted with tacrolimus-free whole blood to produce a series of sample dilutions across a dynamic range of concentrations. The percent recovery was determined by dividing the observed tacrolimus concentration by the expected concentration. Results are provided in Table 2.

TABLE 2
Recovery From Diluted Spiked Sample

| | Expected Value | Observed Value | |
|---|---|---|---|
| % Spiked Sample | (ng/ml) | (ng/ml) | % Recovery |
| 100 | 28.9 | 28.9 | 100.0 |
| 80 | 23.1 | 23.9 | 103.4 |
| 60 | 17.3 | 18.1 | 104.7 |
| 40 | 11.5 | 12.4 | 107.7 |
| 20 | 5.8 | 6.1 | 106.2 |
| 0 | 0.0 | 0.0 | — |

In another experiment, tacrolimus was added to tacrolimus-free whole blood samples as well as patient samples containing tacrolimus to produce specimens with concentrations of 5.0, 10.0, 15.0, and 25.0 ng/ml, as determined by high-performance liquid chromatography/mass spectrometry. Results are shown in Table 3.

TABLE 3
Recovery of Tacrolimus in a CEDIA Assay

| | | Expected Value | Observed Value | |
|---|---|---|---|---|
| Sample | N | (ng/ml) | (ng/ml | % Recovery |
| Sample A | 20 | 5.0 | 5.1 | 101.0 |
| Sample B | 20 | 10.0 | 9.6 | 96.5 |
| Sample C | 20 | 15.0 | 15.8 | 105.2 |
| Sample D | 20 | 25.0 | 25.3 | 101.2 |

### EXAMPLE 10

### Specificity of CEDIA Tacrolimus Assay

Each of four naturally occurring metabolites of tacrolimus, at a concentration of approximately 20 ng/ml, was added separately to a whole blood specimen. Results of each were compared to a control comprising tacrolimus-free whole blood. The cross reactivity of the metabolites was calculated as:
(measured concentration)/20 ng/ml×100%

Results are shown in Table 4.

TABLE 4
Cross-Reactivity with Tacrolimus Metabolites

| Metabolite of | Concentration | Concentration | |
|---|---|---|---|
| Tacrolimus (T) | Tested | Measured | % Cross-Reactivity |
| tacrolimus | 20 ng/ml | 20.0 ng/ml | 100.0 |
| 13-O-demethyl-T | 20 ng/ml | 7.5 ng/ml | 37.7 |
| 15-O-demethyl-T | 20 ng/ml | 0.95 ng/ml | 4.7 |
| 31-O-demethyl-T | 20 ng/ml | 0.98 ng/ml | 4.9 |
| 13,31-0- | | | |
| didemethyl-T | 20 ng/ml | 0.57 ng/ml | 2.9 |

In another experiment, commonly co-administered immunosuppressants were added to whole blood and tested for cross-reactivity. The results are presented in Table 5.

TABLE 5
Cross-Reactivity with Other Immunosuppressants

| | Tested Concentration | |
|---|---|---|
| Immunosuppressant | (ng/ml) | % Cross-Reactivity |
| Cyclosporine | 10,000 | 0.0 |
| Sirolimus (rapamycin) | 30 | 0.7 |
| Mycophenolic acid | 100,000 | 0.0 |
| Prednisone | 100,000 | 0.0 |
| Hydrocorticol | 100,000 | 0.0 |
| Prednisolone | 100,000 | 0.0 |

### EXAMPLE 11

Sensitivity of CEDIA Tacrolimus Assay

The sensitivity of least detectable dose (LDD) was determined by testing 21 tacrolimus-free (negative) whole blood specimens. The LDD, defined as two standard deviations of the tacrolimus concentrations determined in negative specimens, is approximately 2.0 ng/ml.

### EXAMPLE 12

### Method Comparison

A comparison was made between the CEDIA tacrolimus assay described herein and high-performance liquid chromatography/mass spectrometry (LC/MS), microparticle enzyme immunoassay (MEIA), and enzyme multiplied immunoassay (EMIT). The comparison was performed using samples with tacrolimus concentrations ranging from 0 to approximately 30 ng/ml of tacrolimus, obtained from liver and renal transplant patients. Results are shown in Table 6.

TABLE 6
Method Comparison

| CEDIA | Transplant | | | | Correlation |
|---|---|---|---|---|---|
| vs. | Type | n | Slope | Intercept | (r) |
| LC/MS | kidney and | 187 | 1.190 | 0.70 | 0.9643 |
| | liver | | | | |
| LC/MS | kidney | 118 | 1.170 | 0.62 | 0.9667 |
| LC/MS | liver | 69 | 1.193 | 1.04 | 0.9616 |
| MEIA | kidney and | 100 | 1.038 | −0.86 | 0.8476 |
| | liver | | | | |
| MEIA | kidney | 50 | 1.064 | −1.26 | 0.7877 |
| MEIA | liver | 50 | 0.945 | 0.28 | 0.8228 |
| EMIT | kidney and | 93 | 1.019 | 0.12 | 0.9614 |
| | Liver | | | | |
| EMIT | kidney | 70 | 0.998 | 0.32 | 0.9631 |
| EMIT | liver | 23 | 1.100 | −0.70 | 0.9582 |

Although the present invention is fully described herein, the specification and examples are illustrative and not limiting. Other embodiments and modifications may suggest themselves to those skilled in the art without departing from the spirit and scope of the following claims.

1. An immunogen comprising ascomycin derivatized with a carboxymethyl oxime moiety at carbon 22 conjugated to an antigenic carrier molecule and injected into an antibody-producing animal.

2. The immunogen of claim 1 wherein the antigenic carrier molecule comprises a protein, polypeptide, or poly(amino acid).

3. The immunogen of claim 1 wherein the antigenic carrier molecule is chosen from at least one of keyhole limpet hemocyanin and bovine serum albumin.

4. An antibody produced by the immunogen of claim 1 capable of binding to tacrolimus with a cross-reactivity of no more than 5% with each of 15-O-demethyl tacrolimus, 31-O-demethyl tacrolimus, and 13,31-O-didemethyl tacrolimus, and less than 40% cross-reactivity with 13-O-demethyl tacrolimus.

5. The antibody of claim 4 wherein said antibody is a monoclonal antibody.

6. The antibody of claim 4 wherein said antibody has a cross-reactivity of less than 1% with cyclosporin, rapamycin, mycophenolic acid, prednisone, hydrocortisol, and prednisolone.

7. A reagent for use in an immunoassay for tacrolimus comprising ascomycin derivatized with a carboxymethyl oxime moiety at carbon 22 conjugated to a detectable label or solid matrix.

8. The reagent of claim 7 wherein the detectable label comprises an enzyme or enzyme fragment.

9. The reagent of claim 7 wherein the detectable label comprises an enzyme donor fragment capable of undergoing a complementation reaction with an enzyme acceptor fragment that results in restoration of enzymatic activity.

10. The reagent of claim 7 wherein the detectable label comprises β-galactosidase enzyme donor designated ED28.

11. An immunoassay for tacrolimus comprising the antibody of claim 4.

12. An immunoassay for tacrolimus comprising the antibody of claim 5.

13. An immunoassay for tacrolimus comprising the antibody of claim 6.

14. An immunoassay for tacrolimus comprising the reagent of claim 7.

15. An immunoassay for tacrolimus comprising the reagent of claim 8.

16. An immunoassay for tacrolimus comprising the reagent of claim 9.

17. An immunoassay for tacrolimus comprising the reagent of claim 10.

18. A method of measuring the concentration of tacrolimus in a specimen suspected of containing tacrolimus comprising:
(a) reacting the specimen with the antibody of claim 4 in the presence of a labeled reagent comprising at least one of ascomycin, tacrolimus or derivative thereof conjugated to a detectable label; and
(b) measuring the signal associated with the detectable label to determine the concentration of tacrolimus in the specimen.

19. The method of claim 18 wherein the detectable label comprises ascomycin derivatized with a carboxymethyl oxime moiety at carbon 22.

20. The method of claim 18 wherein the detectable label comprises ascomycin derivatized with a carboxymethyl oxime moiety at carbon 22 and conjugated to an enzyme donor fragment.
